(19) United States
(12) Patent Application Publication
(10) Pub. No.: US 2018/0271481 A1
(43) Pub. Date: Sep. 27, 2018

(54) ULTRASOUND DIAGNOSIS SYSTEM, METHOD OF OPERATING ULTRASOUND DIAGNOSIS SYSTEM, AND COMPUTER-READABLE RECORDING MEDIUM

(71) Applicant: OLYMPUS CORPORATION, Tokyo (JP)

(72) Inventor: Takehiro YOSHIMURA, Tokyo (JP)

(73) Assignee: OLYMPUS CORPORATION, Tokyo (JP)

(21) Appl. No.: 15/995,221

(22) Filed: Jun. 1, 2018

Related U.S. Application Data
(63) Continuation of application No. PCT/JP2016/082270, filed on Oct. 31, 2016.

(30) Foreign Application Priority Data
Dec. 3, 2015 (JP) 2015-236611

(51) Int. Cl. A61B 8/00; G06T 7/20; A61B 8/14; A61B 8/12

(52) U.S. Cl. CPC A61B 8/4254; G06T 7/20; G06T 2207/10132; A61B 8/12; A61B 8/14

(57) ABSTRACT

An ultrasound diagnosis system includes: a display configured to display at least part of an ultrasound image generated based on an ultrasound signal received from an ultrasound transducer that transmits an ultrasound to an observation target and receives an ultrasound reflected from the observation target; an input device configured to receive an input of a position on the display to output a position signal; and a controller. The controller is configured to: detect the position from the position signal; determine whether the detected position is within a first area including an area that is part of the ultrasound image displayed on the display and that corresponds to the ultrasound transducer; and when it is determined that the detected position is within the first area, scroll an area of the ultrasound image displayed on the display in accordance with a movement of the detected position.

### CROSS REFERENCES TO RELATED APPLICATIONS

This application is a continuation of PCT international application Ser. No. PCT/JP2016/082270 filed on Oct. 31, 2016 which designates the United States, incorporated herein by reference, and which claims the benefit of priority from Japanese Patent Applications No. 2015-236611, filed on Dec. 3, 2015, incorporated herein by reference.

### BACKGROUND

### 1. Technical Field

The present disclosure relates to an ultrasound diagnosis system, a method of operating the ultrasound diagnosis system, and a computer-readable recording medium.

### 2. Related Art

In the related art, there are known ultrasound diagnosis systems where a radial-type ultrasound transducer is provided at the distal end of an insertion unit to be inserted into the inside of the body of a subject, an ultrasound image is generated on the basis of ultrasound signals received from the ultrasound transducer that transmits ultrasound to an observation target and receives ultrasound reflected from the observation target, and the ultrasound image is observed to diagnose the subject.

Ultrasound images generated by using a radial-type ultrasound transducer are circular images with the ultrasound transducer at a center, and a display unit such as a monitor displays part of the ultrasound image. Therefore, an ultrasound image displayed on the display unit sometimes does not include an area that a user wants to see. In such a case, the user makes a predetermined input through the input device and performs a scrolling operation to scroll the area of the ultrasound image displayed on the display unit. Patent Japanese Laid-open Patent Publication No. 2010-274049 and Japanese Laid-open Patent Publication No. 2010-131396 disclose a configuration using a touch panel as an input device.

### SUMMARY

In some embodiments, an ultrasound diagnosis system includes: a display configured to display at least part of an ultrasound image generated based on an ultrasound signal received from an ultrasound transducer that transmits an ultrasound to an observation target and receives an ultrasound reflected from the observation target; an input device configured to receive an input of a position on the display to output a position signal; and a controller. The controller is configured to: detect the position from the position signal; determine whether the detected position is within a first area including an area that is part of the ultrasound image displayed on the display and that corresponds to the ultrasound transducer; and when it is determined that the detected position is within the first area, scroll an area of the ultrasound image displayed on the display in accordance with a movement of the detected position.

In some embodiments, provided is a method of operating an ultrasound observation device configured to diagnose a subject by observing an ultrasound image generated based on an ultrasound signal received from an ultrasound transducer that transmits ultrasound to an observation target and receives ultrasound reflected from the observation target. The method includes: detecting a position on a display configured to display at least part of the ultrasound image, from a position signal that is output from an input device configured to receive an input of the position; determining whether the detected position is within a first area including an area that is part of the ultrasound image displayed on the display and that corresponds to the ultrasound transducer; and when it is determined that the detected position is within the first area, scrolling an area of the ultrasound image displayed on the display in accordance with a movement of the detected position.

In some embodiments, provided is a non-transitory computer-readable recording medium with an executable program stored thereon. The program is a program for operating an ultrasound observation device configured to diagnose a subject by observing an ultrasound image generated based on an ultrasound signal received from an ultrasound transducer that transmits ultrasound to an observation target and receives ultrasound reflected from the observation target. The program causes the ultrasound observation device to execute: detecting a position on a display configured to display at least part of the ultrasound image, from a position signal that is output from an input device configured to receive an input of the position; determining whether the detected position is within a first area including an area that is part of the ultrasound image displayed on the display and that corresponds to the ultrasound transducer; and when it is determined that the detected position is within the first area, scrolling an area of the ultrasound image displayed on the display in accordance with a movement of the detected position.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram that illustrates a configuration of an ultrasound diagnosis system according to an embodiment of the disclosure;

FIG. 2 is a diagram that illustrates an example of the image presented on a display unit illustrated in FIG. 1;

FIG. 3 is a diagram that illustrates a state during a scrolling operation;

FIG. 4 is a diagram that illustrates a state during a scrolling operation;

FIG. 5 is a diagram that illustrates a state during a rotation operation;

FIG. 6 is a diagram that illustrates a state during a rotation operation;

FIG. 7 is a flowchart that illustrates the outline of a process performed by the ultrasound diagnosis system according to the embodiment of the disclosure;

FIG. 8 is a diagram that illustrates a state of scrolling reset during double tap;

FIG. 9 is a diagram that illustrates a state of scrolling reset during double tap;

FIG. 10 is a diagram that illustrates a state of rotation during a hold-down;

FIG. 11 is a diagram that illustrates a state of rotation during a hold-down;

FIG. 12 is a flowchart that illustrates the outline of the process performed by the ultrasound diagnosis system according to modification 1 of the present embodiment; and

FIG. 13 is a block diagram that illustrates a configuration of an ultrasound diagnosis system according to modification 2 of the present embodiment.

### DETAILED DESCRIPTION

With reference to drawings, an explanation is given below of an embodiment of an ultrasound diagnosis system, a method of operating the ultrasound diagnosis system, and a program for operating the ultrasound diagnosis system according to the disclosure. The disclosure is not limited to the embodiment. In the following embodiment, an ultrasound diagnosis system including a radial-type ultrasound transducer is explained as an example; however, the disclosure is applicable to general-use ultrasound diagnosis systems having a scrolling function for scrolling ultrasound images displayed on a display unit. Therefore, the ultrasound transducer may be not only a radial type but also a convex type or a linear type. Furthermore, in the following embodiment, an explanation is given of for example an ultrasound diagnosis system in which an ultrasound transducer is provided at the distal end of an insertion unit to be inserted into the inside of the body; however, the disclosure is applicable to ultrasound diagnosis systems that observe the inside of the body of the subject from outside the body.

Furthermore, in description of drawings, the same or corresponding components are attached with the same reference numerals as appropriate. Moreover, it should be noted that the drawings are schematic and the relation between components in dimension, the ratio between components, and the like, are sometimes differ from reality. The drawings sometimes contain parts that are different in dimensional relation or proportion.

### Embodiment

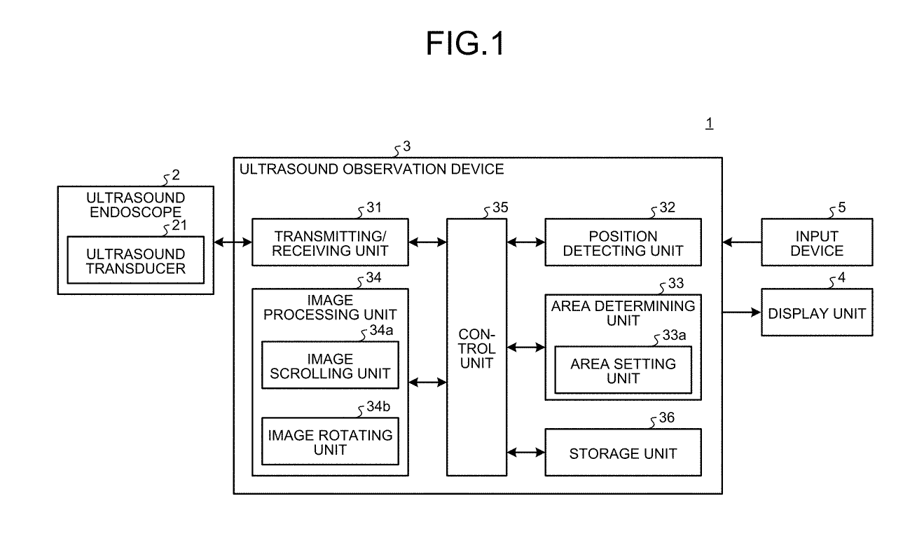

FIG. 1 is a block diagram that illustrates a configuration of an ultrasound diagnosis system according to an embodiment of the disclosure. As illustrated in FIG. 1, an ultrasound diagnosis system 1 includes an ultrasound endoscope 2 that transmits ultrasound to an observation target and receives ultrasound reflected from the observation target; an ultrasound observation device 3 that generates ultrasound images on the basis of ultrasound signals received by the ultrasound endoscope 2; a display unit 4 that displays at least part of the ultrasound image generated by the ultrasound observation device 3; and an input device 5 that receives an input of a position on the display unit 4 and outputs position signals to the ultrasound observation device 3.

At the distal end of the ultrasound endoscope 2, an ultrasound transducer 21 is provided which converts electric pulse signals received from the ultrasound observation device 3 into ultrasound pulses (sound pulses) and emits them to the subject and also converts ultrasound echoes reflected from the subject into electric echo signals (ultrasound signals) represented by changes in a voltage and outputs them. The ultrasound transducer 21 is implemented by using a radial-type sound wave generator. The ultrasound endoscope 2 may cause the ultrasound transducer 21 to conduct scanning mechanically or may cause it to conduct scanning electronically with elements radially arranged as the ultrasound transducer 21 by electronically switching elements for transmitting/receiving or by creasing a delay for each element in transmitting/receiving.

The ultrasound endoscope 2 typically includes an imaging unit including an optical imaging system and an imaging element, and it is inserted into a digestive tract (esophagus, stomach, duodenum, large intestine) or respiratory apparatus (trachea, bronchi) of the subject so as to capture the digestive tract, respiratory apparatus, or their periphery organs (pancreas, gallbladder, bile duct, biliary tract, lymph node, mediastinal organ, blood vessel, or the like). Furthermore, the ultrasound endoscope 2 includes a light guide that guides illumination light emitted to the subject during capturing. The distal end of the light guide reaches the distal end of the insertion unit of the ultrasound endoscope 2 to be inserted into the subject while the proximal end thereof is connected to a light source device that generates the illumination light.

The ultrasound observation device 3 includes a transmitting/receiving unit 31, a position detecting unit 32, an area determining unit 33, an image processing unit 34, a control unit 35, and a storage unit 36.

The transmitting/receiving unit 31 transmits and receives electric signals to and from the ultrasound transducer 21. The transmitting/receiving unit 31 is electrically connected to the ultrasound transducer 21 so that it transmits electric pulse signals to the ultrasound transducer 21 and receives echo signals that are electric reception signals from the ultrasound transducer 21. Specifically, the transmitting/receiving unit 31 generates electric pulse signals on the basis of a predetermined waveform and transmission timing and transmits the generated pulse signals to the ultrasound transducer 21.

The transmitting/receiving unit 31 conducts STC (Sensitivity Time Control) correction to amplify echo signals with a larger receiving depth with a higher amplification factor. After conducting processing such as filtering on amplified echo signals, the transmitting/receiving unit 31 conducts A/D conversion to generate digital high-frequency (RF: Radio Frequency) signals in time domain and outputs them.

The position detecting unit 32 detects a contact position on the display unit 4 in accordance with a position signal output from the input device 5.

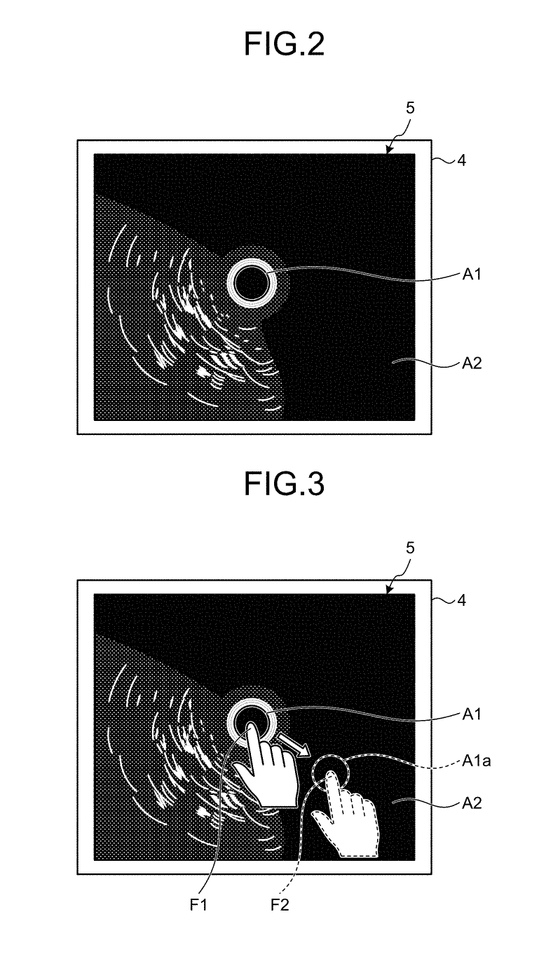

The area determining unit 33 includes an area setting unit 33a that sets a first area and a second area, and it determines whether the position detected by the position detecting unit 32 is within the first area set by the area setting unit 33a. FIG. 2 is a diagram that illustrates an example of the image presented on the display unit illustrated in FIG. 1. As illustrated in FIG. 2, the area setting unit 33a sets, as the first area, an area A1 that is part of the ultrasound image displayed on the display unit 4 and that corresponds to the ultrasound transducer 21 located in the center of FIG. 2. An area A2 other than the area A1 in FIG. 2 is the second area. In FIG. 2, the area that corresponds to the ultrasound transducer 21 on the display unit 4 is identical to the area A1; however, the first area may be any area that includes the area that corresponds to the ultrasound transducer 21. For example, the first area may be an area that includes a balloon located around the ultrasound transducer 21, or it may be an area at the side of the ultrasound transducer 21 from the surface of the living body detected from an ultrasound image. Furthermore, if the ultrasound transducer 21 displayed on the display unit 4 is small, it is preferable that the first area has a size of, for example, equal to or more than 2 cm in diameter for user-friendliness. Moreover, the second area may be any area different from the first area, and multiple areas may be set other than the first area and the second area.

The area setting unit 33a resets the first area used for determination by the area determining unit 33 each time the input device 5 makes an input. Specifically, if the input device 5 has finished inputting a position signal, the area setting unit 33a of the area determining unit 33 sets, as a new first area, an area including the area that is part of the ultrasound image displayed on the display unit 4 when the input of the position signal is finished and that corresponds to the ultrasound transducer 21.

The image processing unit 34 generates ultrasound images that correspond to electric echo signals. Here, as the ultrasound transducer 21 is of a radial type, ultrasound images are circular images with the area that corresponds to the ultrasound transducer 21 as a center. Furthermore, the image processing unit 34 includes an image scrolling unit 34a and an image rotating unit 34b.

If the area determining unit 33 determines that the position detected by the position detecting unit 32 is within the first area and the control unit 35 turns on the scrolling function, the image scrolling unit 34a scrolls the area displayed on the display unit 4, included in the circular ultrasound image, without changing the orientation of the ultrasound image. Here, the scrolling function refers to a function to scroll the position of an ultrasound image displayed on the display unit 4 in accordance with user's operation.

Here, as illustrated in FIG. 2, due to the manner of reflection of ultrasound from the observation target, ultrasound images sometimes have an area (the lower left area in FIG. 2) where echo signals are high and the observation target is easily viewable and an area (the area other than the lower left in FIG. 2) where echo signals are low and the observation target is hard to see. In such a case, a user sometimes scrolls or rotates the area where the observation target is easily viewable to move close to the front (the lower side of the display unit 4) so that it is more viewable.

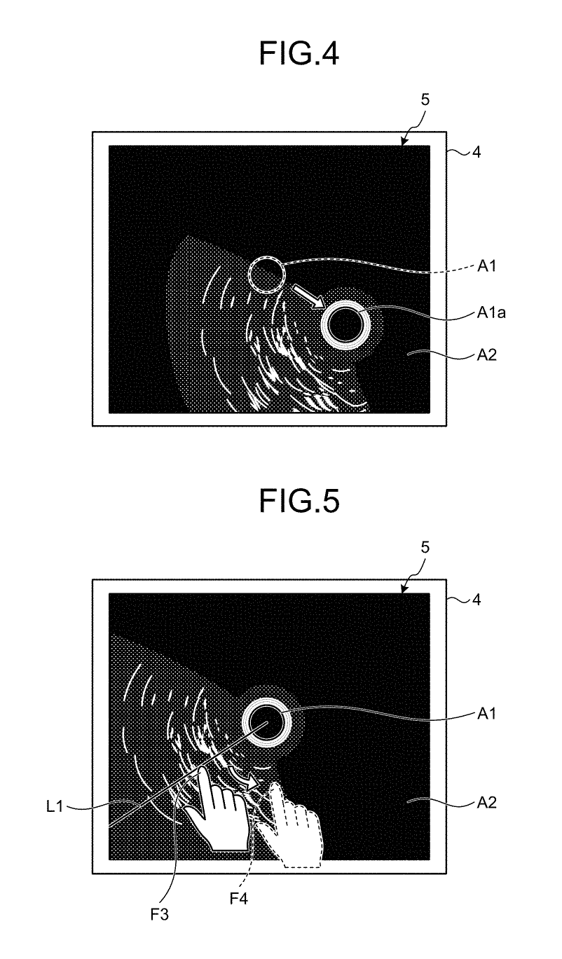

FIGS. 3 and 4 are diagrams that illustrate the states during a scrolling operation. As illustrated in FIG. 3, a finger F1 of the user is in contact with the area A1 of the input device 5, and it moves to the position of a finger F2 indicated by a dashed line in FIG. 3 while it is in contact. Here, as illustrated in FIG. 4, the image scrolling unit 34a scrolls the first area from the area A1 to an area A1a in accordance with the finger. Furthermore, after scrolling, the area A1a is set as a new first area.

If the area determining unit 33 determines that the position detected by the position detecting unit 32 is within the second area and the control unit 35 turns on the rotation function, the image rotating unit 34b rotates the ultrasound image displayed on the display unit 4 with the center of the first area as a rotation center. Here, the rotation function refers to a function to rotate an ultrasound image displayed on the display unit 4 with the center of the first area as a rotation center in accordance with user's operation.

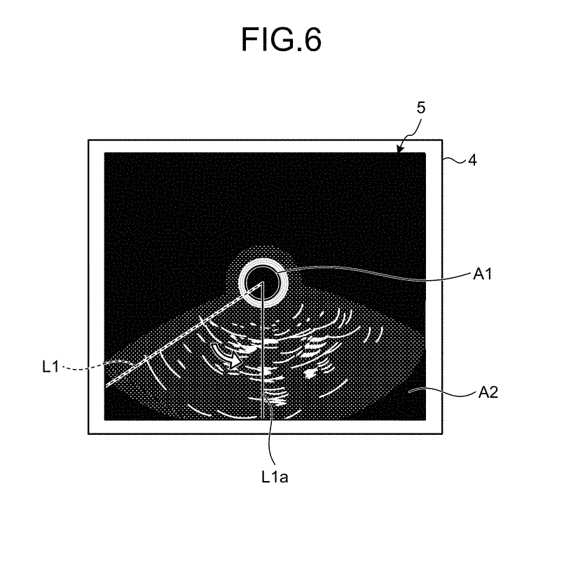

FIGS. 5 and 6 are diagrams that illustrate states during rotation operation. As illustrated in FIG. 5, a finger F3 of the user is in contact with the area A2 of the input device 5, and it moves to the position of a finger F4 indicated by a dashed line in FIG. 5 while it is in contact. Here, as illustrated in FIG. 6, the image rotating unit 34b rotates the ultrasound image with the center of the first area as a rotation center in accordance with the finger so that the direction of a straight line L1 becomes the direction of a straight line L1a.

The control unit 35 performs overall control of the ultrasound diagnosis system 1. The control unit 35 is implemented by using a CPU, various types of arithmetic circuits, or the like, which have calculation and control functions. The control unit 35 reads information, which is saved and stored in the storage unit 36, from the storage unit 36 and executes various types of arithmetic processing related to the method of operating the ultrasound observation device 3, thereby controlling the ultrasound observation device 3 in an integrated manner. For example, the control unit 35 switches on/off the scrolling function and the rotation function. Furthermore, the control unit 35 may be configured by using a CPU, or the like, which is shared by the image processing unit 34.

The storage unit 36 stores data, and the like, including various programs for operating the ultrasound diagnosis system 1, various types of parameters, or the like, needed for operation of the ultrasound diagnosis system 1. The storage unit 36 stores for example the initial position (sound ray number) for the writing position (transmission start position of ultrasound) of ultrasound images.

Furthermore, the storage unit 36 stores various programs including an operation program for implementing the method of operating the ultrasound diagnosis system 1. The operation program may be widely distributed by being recorded in a recording medium readable by a computer such as a hard disk, flash memory, CD-ROM, DVD-ROM, or flexible disk. Furthermore, the above-described various programs may be acquired by being downloaded via a communication network. The communication network mentioned here is implemented by using, for example, an existing public network, LAN (local area network), or WAN (wide area network), and it may be wired or wireless.

The storage unit 36 having the above configuration is implemented by using a ROM (read only memory) that has various programs, and the like, previously installed, a RAM (random access memory) that stores calculation parameters, data, and the like, for processes, and the like.

The display unit 4 displays at least part of a circular ultrasound image generated by using ultrasound transmitted to and received from the ultrasound transducer 21. Part of an ultrasound image displayed on the display unit 4 is changed as appropriate when user's operation is input from the input device 5 and the image scrolling unit 34a and the image rotating unit 34b perform processing in accordance with the input operation. The display unit 4 is configured by using a display panel that is made of liquid crystal, organic EL (electro luminescence), or the like.

The input device 5 is for example a touch panel provided on the display screen of the display unit 4 in an overlapped manner and that detects contact from outside. A position on the display unit 4 is input when a user's finger is brought into contact with the input device 5, and a movement of the position is detected by the position detecting unit 32 when the user's finger is moved in contact with the input device 5. Furthermore, the input device 5 may be a user interface such as mouse or trackball. If the input device 5 is a mouse, the user right-clicks to input a position on the display unit 4, and the user moves (drags) the mouse while right-clicking so that the position detecting unit 32 detects a movement of the position. The input device 5 outputs the received position information as a position signal to the ultrasound observation device 3.

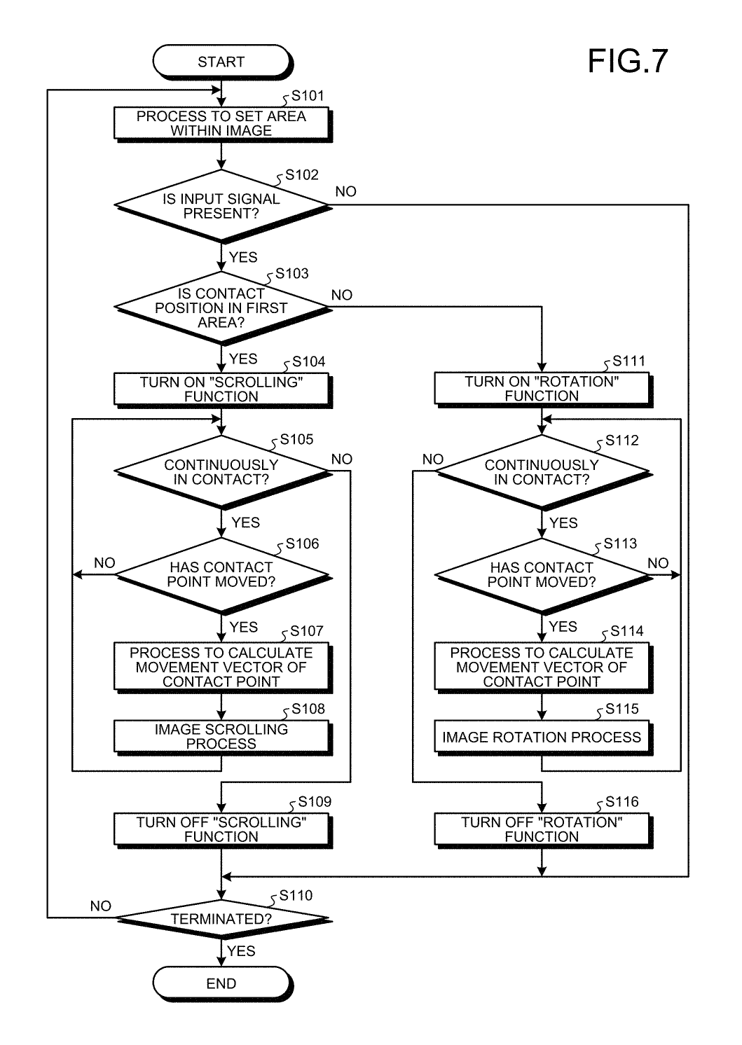

FIG. 7 is a flowchart that illustrates the outline of a process performed by the ultrasound diagnosis system according to the embodiment of the disclosure. First, under the control of the control unit 35, the area determining unit 33 performs the process to set the area that corresponds to the ultrasound transducer 21 as the first area on the screen within the display unit 4. Simultaneously, under the control of the control unit 35, the area setting unit 33a of the area determining unit 33 performs the process to set the area other than the first area within the display unit 4 as the second area (Step S101). In the initial state, for example as illustrated in FIG. 2, the area A1, which is the first area, is located in the center of the display unit 4, and the area around the area A1 is the area A2, which is the second area.

After the areas have been set, the ultrasound diagnosis system 1 is in a stand-by state until user's contact with the input device 5 is detected. Specifically, if user's contact with the input device 5 is detected (Step S102: Yes), the input device 5 outputs a position signal that is information on the contact position to the ultrasound observation device 3. Conversely, if user's contact with the input device 5 is not detected (Step S102: No), termination determination at Step S110 described later is conducted and the process is continued or terminated.

Then, after the position signal is input from the input device 5 to the ultrasound observation device 3, the position detecting unit 32 detects a position on the display unit 4. Furthermore, the area determining unit 33 determines whether the position detected by the position detecting unit 32 is within the first area (Step S103).

If the area determining unit 33 determines that the position detected by the position detecting unit 32 is within the first area (Step S103: Yes), the control unit 35 turns on the scrolling function (Step S104).

If the user is continuously in contact with the input device 5 (Step S105: Yes), the position detecting unit 32 detects a movement of the position. If a user's finger moves and the position detecting unit 32 detects a movement of the position (Step S106: Yes), the control unit 35 calculates a movement vector including the direction and the amount of movement (Step S107) and transmits a calculation result to the image scrolling unit 34a. The image scrolling unit 34a scrolls an area of the ultrasound image displayed on the display unit 4 in accordance with a calculation result of the control unit 35 (Step S108). Then, the process returns to Step S105 and is continued. Conversely, if the position detecting unit 32 does not detect any movement of the position (Step S106: No), the process returns to Step S105 and is continued.

At Step S105, if the user's contact with the input device 5 is terminated (Step S105: No), the control unit 35 turns off the scrolling function (Step S109).

Then, if a predetermined termination command is input (Step S110: Yes), the ultrasound diagnosis system 1 terminates this process. Conversely, if no predetermined termination command is input (Step S110: No), Step S101 is returned. Furthermore, at Step S101, under the control of the control unit 35, the area setting unit 33a of the area determining unit 33 performs the process to reset the area that corresponds to the ultrasound transducer 21 within the display unit 4 as the first area.

If the area determining unit 33 determines that the position detected by the position detecting unit 32 is within the second area (Step S103: No), the control unit 35 turns on the rotation function (Step S111).

If the user is continuously in contact with the input device 5 (Step S112: Yes), the position detecting unit 32 detects a movement of the position. If the user's finger moves and the position detecting unit 32 detects a movement of the position (Step S113: Yes), the control unit 35 calculates a movement vector including the direction and the amount of movement (Step S114) and transmits a calculation result to the image rotating unit 34b. The image rotating unit 34b rotates the ultrasound image displayed on the display unit 4 with the center of the first area as a rotation center in accordance with the calculation result of the control unit 35 (Step S115). Then, the process is returned to Step S112 and is continued. Conversely, if the position detecting unit 32 does not detect any movement of the position (Step S113: No), the process is returned to Step S112 and is continued.

At Step S112, if user's contact with the input device 5 is terminated (Step S112: No), the control unit 35 turns off the rotation function (Step S116). Then, as described above, termination determination is conducted at Step S110 and the process is continued or terminated.

As described above, the ultrasound diagnosis system 1 enables operation to scroll an ultrasound image if the user's contact position with the input device 5 is within the first area. Therefore, the ultrasound diagnosis system 1 allows scrolling operation to be started without inconvenience of input operation.

Furthermore, the ultrasound diagnosis system 1 enables operation to rotate an ultrasound image if the user's contact position with the input device 5 is within the second area. Therefore, the ultrasound diagnosis system 1 makes it possible to selectively use two operations, the scrolling operation and the rotation operation, without inconvenience for performing input operation to switch the operations. Moreover, it is user-friendly that the first area and the second area are set on the basis of the area that corresponds to the ultrasound transducer 21 on the screen within the display unit 4. Thus, users may selectively use the scrolling operation and the rotation operation in an intuitive way.

In the above-described embodiment, an explanation is given of the configuration where the ultrasound diagnosis system 1 performs both the scrolling operation and the rotation operation; however, a configuration may be such that any one of the operations is performed. For example, if the configuration is such that only the scrolling operation is conducted, the scrolling operation may be conducted by touching the first area of the touch panel that is the input device 5, whereby the scrolling operation may be started without inconvenience of input operation. This configuration may be such that when the second area of the input device 5 is touched, a menu screen is displayed.

Modification 1

As the configuration of the ultrasound diagnosis system 1 according to the modification 1 is the same as that of the ultrasound diagnosis system 1 according to the embodiment described in FIG. 1, explanation is omitted. The ultrasound diagnosis system 1 according to the modification 1 is different in processes of the image scrolling unit 34a and the image rotating unit 34b.

The image scrolling unit 34a performs the process to scroll the area displayed on the display unit 4 in accordance with the scrolling operation described in the embodiment. Furthermore, the image scrolling unit 34a performs a scrolling reset process if double tap is input to the input device 5. The scrolling reset process is a process performed by the image scrolling unit 34a to scroll the area of the ultrasound image displayed on the display unit 4 such that the center of the area that corresponds to the ultrasound transducer 21 is located at the center of the display unit 4. Specifically, if the position detecting unit 32 detects positions twice in a row within a predetermined time period and the area determining unit 33 determines that both the positions are within the first area, the area of the ultrasound image displayed on the display unit 4 is scrolled such that the center of the area that corresponds to the ultrasound transducer 21 on the display unit 4 is located at the center of the display unit 4. Here, the predetermined time period during which the position detecting unit 32 detects double tap may be changed by a user.

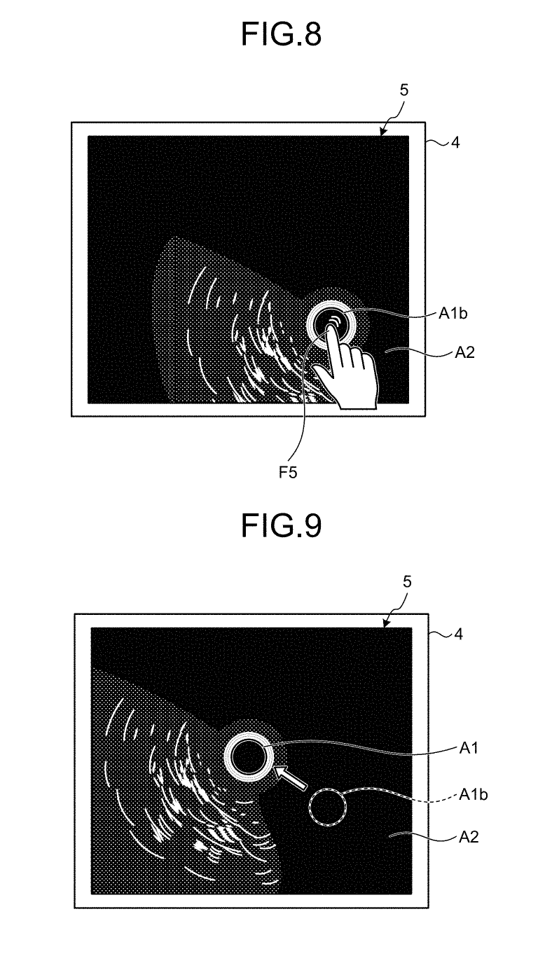

FIGS. 8 and 9 are diagrams that illustrate the state of scrolling reset during double tap. As illustrated in FIG. 8, a user's finger F5 is brought into contact with an area A1b that is the first area of the input device 5 twice in a predetermined time period. At this point, as illustrated in FIG. 9, the image scrolling unit 34a moves the first area from the area A1b to the area A1. This movement may be scrolling, but a configuration may be such that the first area is instantaneously switched from the area A1b to the area A1.

The image rotating unit 34b performs the process to rotate the ultrasound image displayed on the display unit 4 with the center of the first area as a rotation center in accordance with the rotation operation explained in the embodiment. Furthermore, the image rotating unit 34b performs the rotation reset process if double tap is input to the input device 5. The rotation reset process is a process performed by the image rotating unit 34b to return the rotation to the position in the initial state if the image rotating unit 34b has already rotated the ultrasound image displayed on the display unit 4 with the center of the first area as a rotation center. Specifically, if the position detecting unit 32 detects positions twice in a row in a predetermined time period and the area determining unit 33 determines that both the positions are within the second area, the image rotating unit 34b returns the rotated ultrasound image on the display unit 4 to the position in the initial state.

Furthermore, the image rotating unit 34b rotates the ultrasound image displayed on the display unit 4 with the center of the first area as a rotation center if the first area of the input device 5 is held down. Specifically, if the position detecting unit 32 detects the same position during more than a predetermined time period and the area determining unit 33 determines that the position is within the first area, the ultrasound image is rotated with the center of the first area as a rotation center. Furthermore, the predetermined time period during which the position detecting unit 32 makes a determination for a hold-down may be changed by a user.

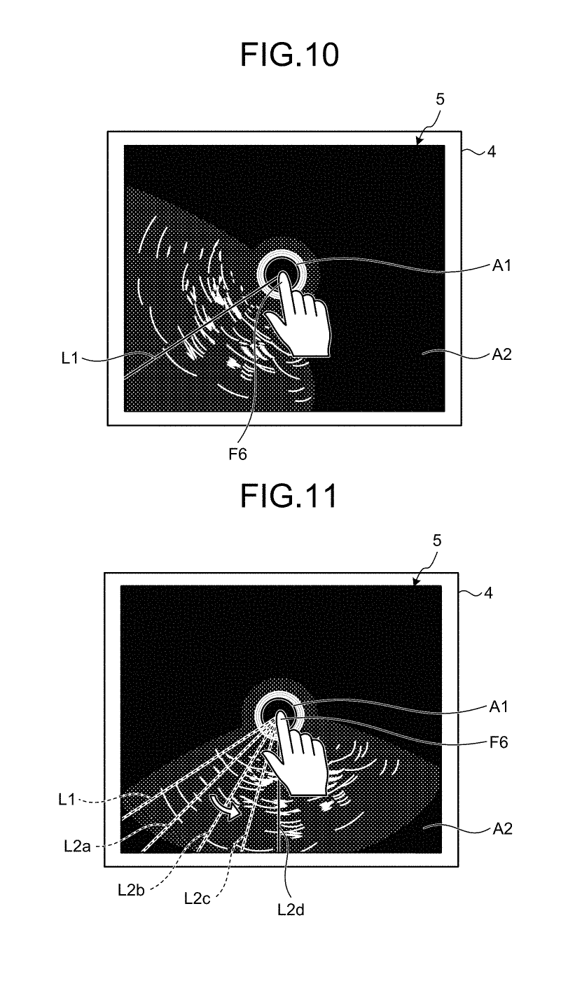

FIGS. 10 and 11 are diagrams that illustrate the state of rotation during a hold-down. As illustrated in FIG. 10, a user's finger F6 is continuously in contact with (holding down) the same position within the area A1, which is the first area of the input device 5, during more than a predetermined time period without moving. Then, as illustrated in FIG. 11, the image rotating unit 34b rotates the ultrasound image in a counterclockwise direction of FIG. 11 at a predetermined speed previously stored in the storage unit 36 such that the direction of the straight line L1 is sequentially moved to a straight line L2a, a straight line L2b, a straight line L2c, and then a straight line L2d. Here, the rotation direction may be in the opposite direction (the clockwise direction of FIG. 11), or a configuration may be such that the rotation speed becomes higher in accordance with the length of a hold-down.

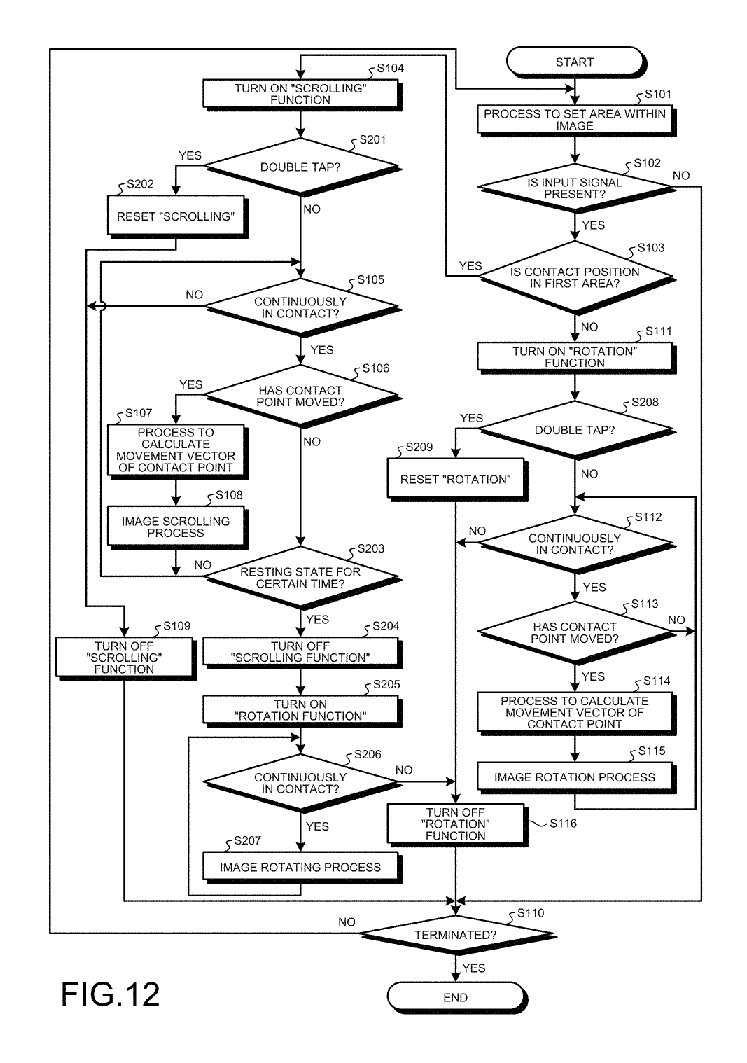

FIG. 12 is a flowchart that illustrates the outline of the process performed by the ultrasound diagnosis system according to the modification 1 of the present embodiment. Explanation is omitted as appropriate for the same process as that in the embodiment.

First, an explanation is given of a case (Step S101 to S104) where the user's contact with the first area of the input device 5 is detected and the control unit 35 turns on the scrolling function. If the position detecting unit 32 terminates position detection once, the position detecting unit 32 detects a position once again within a predetermined time period, and the area determining unit 33 determines that the position is within the area A1, the control unit 35 determines that double tap is input (Step S201: Yes) and performs the scrolling reset process (Step S202).

Then, the control unit 35 turns off the scrolling function (Step S109), termination determination is made at Step S110, and then the process is continued or terminated.

At Step S201, if the control unit 35 determines that double tap is not input (Step S201: No), the control unit 35 determines whether the user is continuously in contact with the input device 5 (Step S105).

If the user is continuously in contact with the input device 5 (Step S105: Yes) and the position detecting unit 32 detects a movement of the position (Step S106: Yes), the same process as that in the embodiment is performed. Conversely, if the control unit 35 determines that the user's contact with the input device 5 is terminated (Step S105: No), the control unit 35 turns off the scrolling function (Step S109), a termination determination is made at Step S110, and then the process is continued or terminated.

If the user is continuously in contact with the input device 5 (Step S105: Yes) and the position detecting unit 32 does not detect any movement of the position (Step S106: No), the control unit 35 determines whether the user's contact with the input device 5 continues during more than a predetermined time period (Step S203). Specifically, the control unit 35 determines whether the position detecting unit 32 has detected the position continuously during more than a predetermined time period.

If the control unit 35 determines that the user is continuously in contact with the input device 5 during more than a predetermined time period (Step S203: Yes), the control unit 35 turns off the scrolling function (Step S204). Then, the control unit 35 turns on the rotation function (Step S205).

Then, the control unit 35 determines whether the user is continuously in contact with the input device 5 (Step S206). If the user is continuously in contact with the input device 5 (Step S206: Yes), the image rotating unit 34b rotates the ultrasound image displayed on the display unit 4 with the center of the first area as a rotation center at a predetermined rotating speed stored in the storage unit 36 (Step S207). Then, the process returns to Step S206 and is continued. Conversely, if it is determined that the user's contact with the input device 5 is terminated (Step S206: No), the control unit 35 turns off the rotation function (Step S116), a termination determination is made at Step S110, and the process is continued or terminated.

Furthermore, at Step S203, if the control unit 35 determines that the user's contact with the input device 5 does not continue during more than a predetermined time period (Step S203: No), the process is returned to Step S105 and is continued.

Next, an explanation is given of a case where the area determining unit 33 determines that the position detected by the position detecting unit 32 is within the second area (Step S103: No) and the control unit 35 turns on the rotation function (Step S111). If the position detecting unit 32 terminates position detection once, the position detecting unit 32 detects a position once again within a predetermined time period, and the area determining unit 33 determines that the position is within the second area, the control unit 35 determines that double tap is input (Step S208: Yes) and performs the rotation reset process (Step S209).

Then, the control unit 35 turns off the rotation function (Step S116). Furthermore, as described above, a termination determination is made at Step S110, and the process is continued or terminated.

At Step S208, if the control unit 35 determines that double tap is not input (Step S208: No), the same process as that in the embodiment is performed.

As described above, the ultrasound diagnosis system 1 enables operation to scroll an ultrasound image if the user's contact position with the input device 5 is the first area. Therefore, the ultrasound diagnosis system 1 allows scrolling operation to be started without inconvenience of input operation.

Furthermore, the ultrasound diagnosis system 1 enables operation to rotate an ultrasound image if the user's contact position with the input device 5 is the second area. Therefore, the ultrasound diagnosis system 1 makes it possible to selectively use two operations, the scrolling operation and the rotation operation, without inconvenience for performing input operation to switch the operations. Moreover, as the scrolling reset process and the rotation reset process during double tap and a switchover between operations during a hold-down are possible, user's operability is improved.

Furthermore, according to the above-described modification 1, a configuration may be such that a specific process is performed when the second area is held down. For example, a configuration may be such that, when the second area is held down, the rotation function is switched to the scrolling function and the center of the area that corresponds to the ultrasound transducer 21 on the display unit 4 is moved closer to the input position at a predetermined speed stored in the storage unit 36.

Modification 2

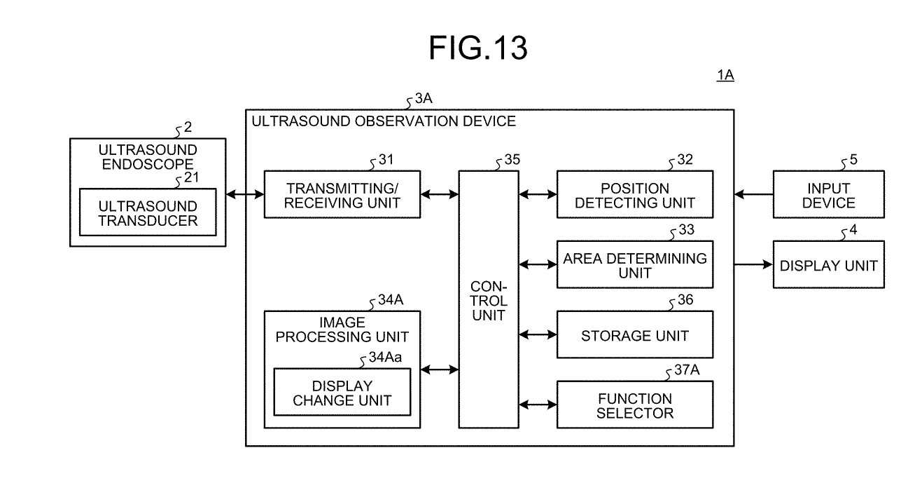

FIG. 13 is a block diagram that illustrates a configuration of an ultrasound diagnosis system according to modification 2 of the present embodiment. As an ultrasound diagnosis system 1A according to the modification 2 has the same configuration as that of the ultrasound diagnosis system 1 according to the embodiment except for the configuration of part of an ultrasound observation device 3A, explanation is omitted as appropriate.

The ultrasound observation device 3A includes the transmitting/receiving unit 31, the position detecting unit 32, the area determining unit 33, an image processing unit 34A, the control unit 35, the storage unit 36, and a function selector 37A.

The function selector 37A selects any one of the scrolling function and the rotation function in accordance with a determination result of the area determining unit 33 under the control of the control unit 35. Specifically, the function selector 37A selects the scrolling function if the area determining unit 33 determines that the position detected by the position detecting unit 32 is within the first area, and the function selector 37A selects the rotation function if the area determining unit 33 determines that the position detected by the position detecting unit 32 is within the second area.

The image processing unit 34A includes a display change unit 34Aa. The display change unit 34Aa changes the ultrasound image displayed on the display unit 4 in accordance with the function selected by the function selector 37A. Specifically, the display change unit 34Aa scrolls the position of the ultrasound image displayed on the display unit 4 in accordance with user's operation if the scrolling function is selected by the function selector 37A, and the display change unit 34Aa rotates the ultrasound image displayed on the display unit 4 with the center of the first area as a rotation center if the rotation function is selected by the function selector 37A.

The ultrasound diagnosis system 1A enables operation to scroll an ultrasound image if the user's contact position with the input device 5 is within the first area. Therefore, the ultrasound diagnosis system 1A allows scrolling operation to be started without inconvenience of input operation. Furthermore, the ultrasound diagnosis system 1A enables operation to rotate an ultrasound image if the user's contact position with the input device 5 is within the second area. Thus, the ultrasound diagnosis system 1A makes it possible to selectively use two operations, the scrolling operation and the rotation operation, without inconvenience for performing input operation to switch the operations.

Furthermore, the configurations of the above-described embodiment and the modification 2 are examples, and location and processing of each functional unit may be changed as appropriate as long as a configuration is such that functions such as the scrolling operation and the rotation operation are selectively used in accordance with the user's contact position with the input device 5.

Furthermore, according to the above-described embodiment, a configuration may be such that the outline of the first area displayed on the display unit 4 is highlighted. A configuration may be such that highlighting is displaying a line on the outline of the first area. Furthermore, a configuration may be such that the line is flashed. Moreover, the entire first area may be painted in any color.

Furthermore, according to the above-described embodiment, operation may be restricted to handle incorrect operation, or the like. For example, during the scrolling operation, operation of the image scrolling unit 34a may be restricted to prevent the center of the area that corresponds to the ultrasound transducer 21 on the display unit 4 from being out of the display unit 4.

Furthermore, in the above-described embodiment, an explanation is given based on a live display, that is, the ultrasound endoscope 2 generates ultrasound images in sequence on the basis of sequentially acquired ultrasound signals and displays them on the display unit 4; however, the configuration according to the above-described embodiment is applicable to a freeze display that is displayed when a user specifies an ultrasound image in desired timing.

Furthermore, according to the above-described embodiment, the input device 5 may be an eye sensor. If the input device 5 is an eye sensor, for example when a user continuously watches the same position on the first area for more than a certain period of time, the position detecting unit 32 detects the position. Then, if the user's line of sight moves, the image scrolling unit 34a scrolls the area of the ultrasound image displayed on the display unit 4 in accordance with the movement. Furthermore, the scrolling function may be terminated when a movement of the user's line of sight stops for more than a predetermined time period, or when the user closes the eye.

According to some embodiments, it is possible to implement an ultrasound diagnosis system, a method of operating the ultrasound diagnosis system, and a program for operating the ultrasound diagnosis system with which a scrolling operation may be started without inconvenience of input operation.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound diagnosis system comprising:
a display configured to display at least part of an ultrasound image generated based on an ultrasound signal received from an ultrasound transducer that transmits an ultrasound to an observation target and receives an ultrasound reflected from the observation target;
an input device configured to receive an input of a position on the display to output a position signal; and
a controller, wherein
the controller is configured to:
detect the position from the position signal;
determine whether the detected position is within a first area including an area that is part of the ultrasound image displayed on the display and that corresponds to the ultrasound transducer; and
when it is determined that the detected position is within the first area, scroll an area of the ultrasound image displayed on the display in accordance with a movement of the detected position.

2. The ultrasound diagnosis system according to claim 1, wherein
the controller is further configured to:
determine whether the detected position is in a second area that is an area different from the first area; and
when it is determined that the detected position is within the second area, rotate the ultrasound image displayed on the display with a center of the first area as a rotation center in accordance with a movement of the detected position.

3. The ultrasound diagnosis system according to claim 1, wherein the first area is an area of the ultrasound transducer area.

4. The ultrasound diagnosis system according to claim 1, wherein when an input of the position signal from the input device is finished, the controller is configured to set, as a new first area, an area including an area that is part of the ultrasound image displayed on the display at a time when the input of the position signal is finished and that corresponds to the ultrasound transducer.

5. The ultrasound diagnosis system according to claim 1, wherein the input device is a touch panel that is provided on a display screen of the display in an overlapped manner and that detects contact from outside.

6. The ultrasound diagnosis system according to claim 1, wherein
the ultrasound transducer is of a radial type, and
the ultrasound image is a circular image whose center is an area that corresponds to the ultrasound transducer.

7. The ultrasound diagnosis system according to claim 1, wherein when the position is detected twice in a row within a predetermined time period and it is determined that both the detected positions are within the first area, the controller is configured to scroll an area of the ultrasound image displayed on the display to locate a center of the area that corresponds to the ultrasound transducer at a center of the display.

8. The ultrasound diagnosis system according to claim 2, wherein when the position is detected, it is determined that the detected position is within the first area, and the position is continuously detected during more than a predetermined time period, the controller is configured to rotate the ultrasound image displayed on the display with a center of the first area as a rotation center.

9. The ultrasound diagnosis system according to claim 1, wherein an outline of the first area displayed on the display is highlighted.

10. A method of operating an ultrasound observation device configured to diagnose a subject by observing an ultrasound image generated based on an ultrasound signal received from an ultrasound transducer that transmits ultrasound to an observation target and receives ultrasound reflected from the observation target, the method comprising:
detecting a position on a display configured to display at least part of the ultrasound image, from a position signal that is output from an input device configured to receive an input of the position;
determining whether the detected position is within a first area including an area that is part of the ultrasound image displayed on the display and that corresponds to the ultrasound transducer; and
when it is determined that the detected position is within the first area, scrolling an area of the ultrasound image displayed on the display in accordance with a movement of the detected position.

11. The method of operating the ultrasound observation device according to claim 10, further comprising:
determining whether the detected position is in a second area that is an area different from the first area; and
when it is determined that the detected position is within the second area, rotating the ultrasound image displayed on the display with a center of the first area as a rotation center in accordance with a movement of the detected position.

12. The method of operating the ultrasound observation device according to claim 10, wherein the first area is an area of the ultrasound transducer area.

13. A non-transitory computer-readable recording medium with an executable program stored thereon, the program operating an ultrasound observation device configured to diagnose a subject by observing an ultrasound image generated based on an ultrasound signal received from an ultrasound transducer that transmits ultrasound to an observation target and receives ultrasound reflected from the observation target, the program causing the ultrasound observation device to execute:
detecting a position on a display configured to display at least part of the ultrasound image, from a position signal that is output from an input device configured to receive an input of the position;
determining whether the detected position is within a first area including an area that is part of the ultrasound image displayed on the display and that corresponds to the ultrasound transducer; and
when it is determined that the detected position is within the first area, scrolling an area of the ultrasound image displayed on the display in accordance with a movement of the detected position.

14. The non-transitory computer-readable recording medium according to claim 13, wherein
the program causes the ultrasound observation device to execute:
determining whether the detected position is in a second area that is an area different from the first area; and
when it is determined that the detected position is within the second area, rotating the ultrasound image displayed on the display with a center of the first area as a rotation center in accordance with a movement of the detected position.

15. The non-transitory computer-readable recording medium according to claim 13, wherein the first area is an area of the ultrasound transducer area.
